(19) United States
(12) Patent Application Publication
(10) Pub. No.: US 2008/0299150 A1
(43) Pub. Date: Dec. 4, 2008

(54) METHOD AND SYSTEM FOR PROCESSING IMMUNO-COMPETENT CELLS IN VIEW OF AN ANTIVIRAL THERAPY, AND RELATED THERAPEUTIC PROCESS

(75) Inventors: Patrick Rambaud, Montreal (CA); Jean-Pierre Lablanchy, Paris (FR)

Correspondence Address: YOUNG & THOMPSON, 209 Madison Street, Suite 500, ALEXANDRIA, VA, 22314, US

(73) Assignee: SANKHIA CORPORATION, Plattsburg, NY (US)

(21) Appl. No.: 12/113,961

(22) Filed: May 2, 2008

Related U.S. Application Data
(60) Provisional application No. 60/924,295, filed on May 8, 2007.

(51) Int. Cl. A61K 39/00; C12N 5/06; C12M 1/00; A61P 31/00; A61K 35/12

(52) U.S. Cl. 424/206.1; 435/375; 435/374; 424/93.71; 424/93.21; 424/184.1; 424/208.1; 424/231.1; 424/207.1; 435/307.1

(57) ABSTRACT

A method for processing immuno-competent cells in view of an antiviral therapy on a human or animal subject, includes:

a step for collecting immuno-competent cells on the human or animal subject, and

a step for processing the collected immuno-competent cells, so as to stimulate at least a part of the immuno-competent cells, by presenting a muted viral particle to the cells, in view of a reinjection of the stimulated cells into the human or animal subject.

The present invention concerns a method for processing immuno-competent cells in view of a antiviral therapy. It also concerns a system implementing said method, and a related therapeutic method.

Searching and developing antiviral treatment methods is presently crucial and constitutes a major health challenge. Important research efforts are presently done to get antiviral vaccines. But, it makes clear that developing such vaccines is particularly difficult, time-consuming and costly, furthermore with many alea on their real effectiveness in the case of frequently muting viruses.

Viruses implement strategies permitting them to inhibit the immunity response directed against them. In the case of retroviruses, Thierry Heidmann and his team have identified a domain having an immunosuppressive activity, and the amino acids of the related protein which is responsible for the activity.

Document WO2004/087748 thus discloses a protein or polypeptide having a fusogenic activity, sequences of nucleic acid encoding this protein and pharmaceutical compositions obtained from this protein.

A rule is proposed to pass from a positive immunosuppressive domain to a negative immunosuppressive domain, while preserving the infectious function, and thus the épitopes of the muted viruses.

Document WO 2005/095442 discloses a sequence of polypeptides which is implicated in the modulation of the immunosuppressant effect of viral proteins. This polypeptide, which has a sequence of 7 to 20 amino acid residues, is able to modulate the immunosuppressive properties of a viral protein or of a fragment of this viral protein, against the host in which it is expressed when it is substituted to the homologous of the viral protein or of its fragment, this polypeptide comprising a minimal consensus amino acid sequence.

It thus has been demonstrated on different model retroviruses that replacing the two incriminated amino acids by others causes the neutralization of the virus that is no more capable to colonize the host, on the contrary of the savage virus.

The aim of the present invention is to propose an innovative approach in the viral therapeutic strategy, that would particularly allow a drastic reduction in the reaction times for establishing a therapy against a major viral aggression and a pandemia.

This objective is reached with a method for processing immuno-competent cells in view of an antiviral therapy on a human or animal subject, comprising:

a step for collecting immuno-competent cells on said human or animal subject, and

a step for processing said collected immuno-competent cells, so as to stimulate at least a part of said immuno-competent cells, by presenting a muted viral particle to said cells, in view of a reinjection of said stimulated cells into said human or animal subject.

The invention consists in directly activating the lymphocytes or the dendritic cells in vitro, and then re injecting them into the patient in an autologous way.

The processing method according to the invention can be directly implemented with no delay between collecting cells, stimulating said cells, and reinjecting them into the patient.

But, in a particular embodiment of the invention, the processing method can also include:
a step for cryogenically preserving collected immuno-competent cells, and
a step for defrozing said cryogenically preserved cells, in view of processing said cells.

In another embodiment of the invention, the processing method can also include:
a step for cryogenically preserving processed and stimulated immuno-competent cells, and
a step for defrozing said cryogenically preserved stimulated cells, in view of reinjecting said cells.

In these two embodiments, the preserved immuno-competent cells can be stored into a cell-bank.

Document U.S. Pat. No. 6,415,201 discloses a method and a system for managing batches of cells from the haematopoietic system that have been collected in view of a deferred use. Each batch is conditioned and stored into a cryogenic storage site, and then is transferred on request from this storage site to a cell treatment centre. At different steps of the process, personal data related to the subject from whom said cells are collected and to collecting condition are collected.

Document US 2004/0138822 discloses a method and a system for managing batches of immuno-competent cells which have been collected from a human or animal subject, with the aim to constitute a personal library of immunity information that are stored into the already collected immuno-competent cells.

The method according to the invention can be implemented for treating viral diseases such as flu, avian flu, AIDS (HIV) or herpes. The human retroviruses include HTLV and HIV.

The concept consists in providing the organism with an additional chance to survive to an attack by micro organisms, in particular viral, retroviral, oncongenous hexogenous and/or endogenous micro organisms.

As the organism may be simultaneously attacked by several ways, due to the immuno-depression, it is possible to multiply the lymphocytes in vitro in order to inform them by one or more antigens, together or separately, and/or in an iterative way.

The presentation of preserved naive lymphocytes can be of interest, but may risk to have this additional population follow the same way as the lymphocytes in place into the organism and facing the micro-organic attack.

Being able to present in vitro a muted antigen having a negative immunodepressive function allows the lymphocyte to resist to the attack of this micro organism in vivo and to multiply by 10 its ability to produce antibodies.

With reference to Thierry Heidmann's works, the mutation concerns 4 amino acids which are the specifically positive immunodepressive domain of the virus. The mutation of 2 to 4 amino acids is the necessary and sufficient requirement for the virus to loose its immunodepressive function while preserving its infectious and antigenic power.

This concept could be applied to viruses in general, to retroviruses and to the flu virus, and particularly to the avian flu virus H5N1 and to any expected and presently expectable form of mutation.

Lymphocytes that are beforehand informed after an in vivo stimulation, could resist to the incriminated micro-organism, but also destroy cells that have been infected by the savage micro-organism.

The capability of an amplification from the preserved sample permits to significantly increase the capacities of resistance to one or more simultaneous or time-spaced attacks.

The present invention can also be applied to retroviruses included in the genome along the species history, as theses retroviruses present a positive immunodepressive domain leading to immuno-depression and facilitating the evolution of an oncogenous process.

For grafts, it is possible to present to lymphocytes the domain that is specifically positive immunodepressive to lymphocytes, in order to modulate the immunizing reaction of the organism against a graft.

It could be possible to better to control both compatible grafts and exogenous grafts, provided that the lymphocytes have been beforehand informed against the noxious presence of viruses in the exogenous graft. It concerns for example the pork which presents numerous retroviruses which are dangerous for a grafted subject, which is the main reason of the present practical absence of exogenous grafts.

It could be possible to use contaminated grafts in the same way, after having beforehand provided the organism with autologous lymphocytes that have been beforehand informed, thus permitting to solve the problem of the presence of a pathogen micro-organism into the graft.

Enhancing the resistance of the organism to a micro-organic attack will get more efficient any conventional antibiotic and/or chemotherapeutic treatment or any other treatment aimed to control this attack.

This lymphocyte in vitro preparation provides an additional arm to the arsenal of the anti-cancerous individual active immunotherapy.

When the retroviruses which are present in the genome of a subject can be identified, it would then be possible to <<vaccine>> the lymphocytes in vitro against these active retroviruses, in order to achieve an effective prevention of their possible pathogen activation in an immuno-depressive context, whatever is the cause.

For anti-cancerous treatments, being able to use the lymphocytes that have been processed in vitro against the positive immunodepressive domains, will enhance the results of the conventional chemotherapy.

As it is possible to mute any micro organism presenting this positive immunodepressive domain strategy, one can expect achieving a therapeutic strategy in the case of a so called “bacteriologic” war, in fact a strategy related to the use, for war goals, of any micro-organism which is pathogen for humans and/or animals.

The present invention leads to the following proposals:
1) a method for treating attacks by micro-organisms having this immunodepressive arm easing their expansion into the organism;
2) a method for treating exogenous banal but strongly oncogenous viruses (CMV EBV, for example) or pandemic viruses such as the flu virus or a mutation of the avian flu;
3) a method for preventing positive immunodepressive endogenous domains from “awaking” which would lead to a cancerising process.
4) an immunomodulation method for facilitating organ grafts, and permitting to enhance the safety conditions in the field of grafts that have been contaminated by micro-organisms;
5) a method for enhancing the objective results obtained by presently used conventional treatments or by strategies that could be developed in the future.

Among cells collected from a subject and stored within the method according to the invention, three types of cells can be observed:
dendritic cells,
B lymphocytes,
macrophages.

Dendritic cells are specialized in “appreting” and presenting antigens, and is defined in a morphologic, phenotypic and functional point of view. These cells can be immature or mature, said two states corresponding to two distinct functional situations. In the immature state, the dendritic cell is capable to phagocyte and “apprete” antigens. In the mature state, it presents the antigen via the molecules of the type I or type II histo compatibility major complex (CMH), and activates the T lymphocytes by means of the costimulation molecules (CD80,86, . . . ).

To achieve the treatment of the collected immuno-competent cells, three modes can be provided:
an in vitro stimulation of lymphocytes with dendritic cells from the collected batch, and their re injection in the patient,
a fusion of dendritic cells and the muted virus, and then their re injection in the patient, with the aim of stimulating the lymphocytes in vivo,
an initial in vitro costimulation of lymphocytes, and then their re injection in the patient, followed by a succession of “boosters” according to a predetermined schedule;

In a first approach, the muted virus can be produced as such, as viral particle. Then it is inactivated by known inactivation processes, and then incubated with dendritic cells derived from the cryogenised batch.

In a second approach, only the genome of the viral particle is introduced. It is transfected into dendritic cells, so that its proteins are expressed, leading to the production of peptides.

This transfection can be done in several ways:

via a viral vector, like an adenovirus, a lentivirus, an herpetic amplicon,

as a plasmide,

as an ARN.

Transfecting the cell causes it to be cut into peptides which are presented by the molecules of the HLA complex t the surface of the dendritic cell. The complex is recognized by the antigen receptor of the T lymphocytes.

T lymphocytes (cytotoxic) recognize the same peptide—derived from the muted virus—. They receive an activation signal which lead them to multiply, thus contributing to the increase of lymphocytes.

When these lymphocytes are injected into the patient's blood stream and they meet a cell having the same peptide, then they destroy this cell.

The method according to the invention can also be proposed for tumors that could disappear following the information they have received, by means of retroviruses that are present in our genetic patrimony and that responsible for apparition of cancers following an immuno-senescence and thus an immunodeficiency or an immunodepressive episode.

Applications of the method according to the invention may also include hepatites, including some types of tumors, herpes, AIDS virus, avian flu, flu viruses, SARS virus, and Ebola virus.

Generally, the method according to the invention should concern any type of virus which could be transformed into a muted virus, keeping a part of its viral properties.

The activation could also be done after selection of lymphocytes or dendritic cells or any white globule. Cells that have already be in presence of the disease, particularly the memory cells, but also cells that present to the antigen and have a major role.

The whole cell treatment chain, from cell defrosting to selecting, activating or expanding, can be automatized and controlled by an expert system which coordinates the various automats provided for implementing the conventional handling sequences.

Advantages and characteristics of the invention will be better understood through the detailed description of a non-limitative embodiment, with the attached drawings:

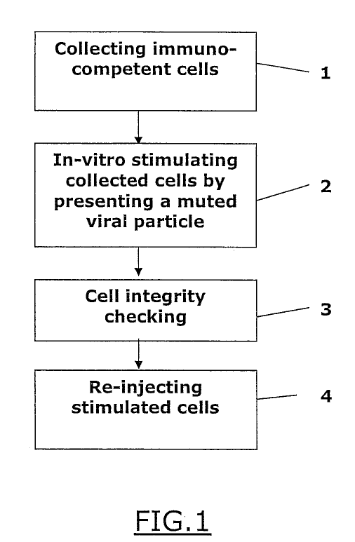

FIG. 1 is a diagram of the steps implemented in a direct embodiment and use of the processing method according to the invention;

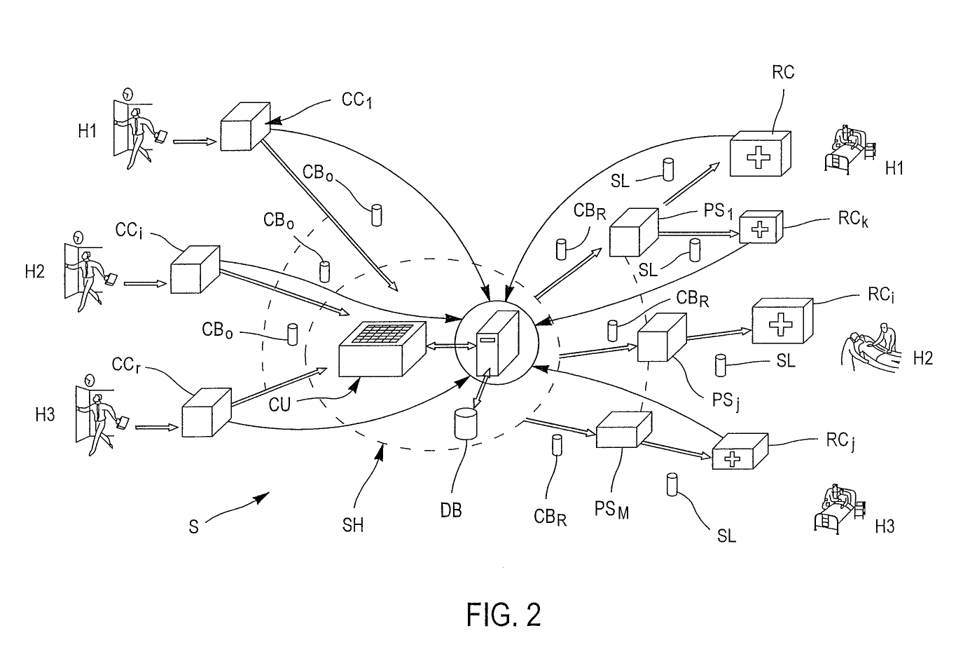

FIG. 2 is a synoptic of a system according to the invention, for managing and processing immuno-competent cells;

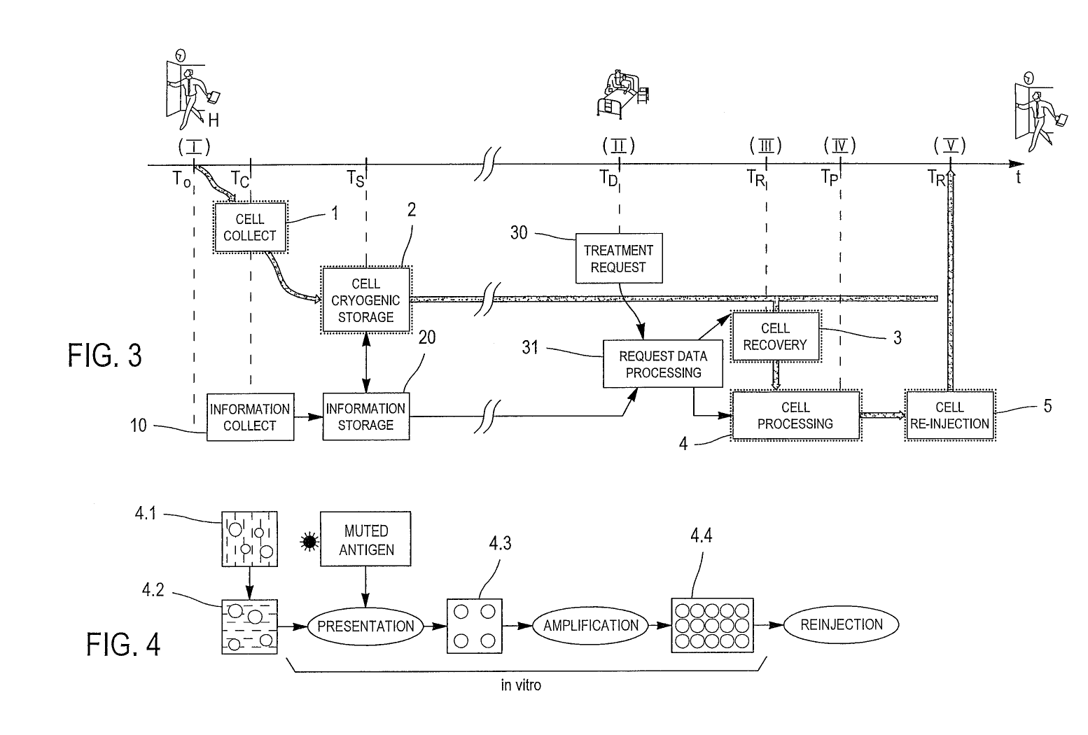

FIG. 3 features the steps of a management and processing method according to the invention; and

FIG. 4 features steps for processing a batch of immuno-competent cells, within the invention.

With reference to FIG. 1, immuno-competent cells are collected (step 1) from a patient. These collected cells are processed (step 2) so as to stimulate at least a part of said immuno-competent cells, by presenting a muted viral particle to said cells. After a checking step (3) for cell integrity, the stimulated cells are reinjected (step 4) into the patient.

With reference to FIG. 2, an example of embodiment of a system S according to the invention for managing and processing immuno-competent cells is described This system comprises a central storage and management site SH, a plurality of collecting sites or centres CC1, . . . ,CCi, . . . CCN, a plurality of cell treatment sites PS1, . . . ,PSj, . . . PSM, and a plurality of re injection sites RC1, . . . ,RCk, . . . RCj.

These various sites are connected to the central storage and management site SH via communication networks and an air and/or land logistic structure, for conveying immuno-competent cells batches between said concerned sites.

The collecting centres CC1, . . . ,CCi, . . . ,CCn are provided for collecting from human subjects H1-H3 immuno-competent blood cells, particularly lymphocytes. These collected cells are conditioned in batches Cbo that are identified and encoded and then conveyed to a cryogenic storage unit CU located within the central storage and management site SH. It should be noted that a same batch of cells collected from a subject can be fractioned in a plurality of batches for management and safety purposes.

A transmission of information relating to the subject and to the collection conditions is associated to each collection and storage of a batch of cells Cbo, towards an information system comprising a management server MS and data base DB.

In response to a treatment request for a subject emitted from a healthcare centre provided with re injection or recovery sites, a batch CB_{R} of cells that were beforehand collected from this subject is conveyed towards a cell processing site PSj. These cells are then processed according to a processing method that will be described below, in order to deliver a batch of stimulated cells S_{L} which is then transmitted to the healthcare centre RCk to be recovered to the subject as a patient. It should be noted that one can propose a configuration wherein the cell processing site is provided inside the healthcare centre. Furthermore, the collecting site and the processing site can also be located in a same place.

The various steps of an example of implementation of the treatment method according to the invention will now be described in more details, with reference to FIG. 3, in the case or a subject suffering from a viral disease.

A healthy human subject H decides to participate in a cell collecting program implementing the management and processing method according to the invention. At an instant To, he/she undergoes a operation (1) for collecting blood cells that are then conditioned and conveyed (2) to a cryogenic storage unit? In the same sequence, at instant Tc, information related to the collected subject and to his/her health condition are collected (10) and transmitted to the central storage and management centre to be stored (20) into a secured data base.

Several months or years later, at an instant TD, this subject suffers from a viral disease which is identified through medical test. The subject or his/her relatives transmit (30) to the central storage and management site SH a request for recovering the batch of cells that had been collected at the instant To on this subject now a patient. The information system in the central site SH processes (31) the request data and identifies a concerned batch of cells and its location.

At an instant T_{R}, this batch of cells is removed (3) from the cryogenic storage unit, and then defrosted. Cells are then submitted (4), at an instant Tₚ, to a process which is specific for the type of virus implicated in the concerned subject's disease. At the end of this processing phase, the stimulated cells that have been obtained are re injected (5), at an instant T_{R}, into the subject, according to a predetermined therapeutic protocol.

During the cell processing phase 4 schematically illustrated in FIG. 4 and which is done in vitro, immuno-competent cells, that were initially in a frozen state 4.1, are brought back to ambient state 4.2. An antigen of the muted virus is then presented to all or part of the immuno-competent cells, so as to obtain stimulated cells which will be capable to recognize in vivo cells that are infected by the virus and to destroy it. A amplification step is done beforehand in order to increase the number of stimulated cells which are then re-injected into the patient.

Of course, the invention is not limited to the above-described examples and numerous arrangements can be provided to said examples without departing from the scope of the invention.

1. A method for processing immuno-competent cells in view of an antiviral therapy on a human or animal subject, comprising:
a step for collecting immuno-competent cells on said human or animal subject, and
a step for processing said collected immuno-competent cells, so as to stimulate at least a part of said immuno-competent cells, by presenting a muted viral particle to said cells, in view of a re injection of said stimulated cells into said human or animal subject.

2. The method of claim 1, further including:
a step for cryogenically preserving collected immuno-competent cells, and
a step for defrozing said cryogenically preserved cells, in view of processing said cells.

3. The method of claim 1, further including:
a step for cryogenically preserving processed and stimulated immuno-competent cells, and
a step for defrozing said cryogenically preserved stimulated cells, in view of reinjecting said cells.

4. The method of claim 2, wherein the preserved immuno-competent cells are stored into a cell-bank.

5. The method of claim 1, wherein the collected immuno-competent cells comprise dendritic cells, B lymphocytes and macrophages.

6. The method of claim 5, wherein the immuno-competent cell processing step comprises an in vitro stimulation of lymphocytes by dendritic cells deriving from collected immuno-competent cells and to which a muted viral particle has been presented.

7. The method of claim 6, wherein the viral particle is beforehand inactivated before being incubated with dendritic cells.

8. The method according to claim 6, wherein the immuno-competent cells processing step comprises fusing dendritic cells from collected immuno-competent cells with a muted virus (or a muted viral particle), in view of their reinjection into the human or animal subject so as to in vivo stimulate lymphocytes.

9. The method of claim 8, wherein it further comprises a phase for transfecting the muted viral particle into dendritic cells from the collected cells so that its proteins are expressed and lead to cutting out said dendritic cells into peptides.

10. The method according to claim 1, wherein the immuno-competent cells processing step includes the step of initially in vitro co-stimulating lymphocytes before their re injection into said human or animal subject, followed by one ore more further injections (“boosters”).

11. The method according to claim 1, applied to flu viruses.

12. The method according to claim 1, applied to HIV virus.

13. The method according to claim 1, applied to the herpes virus.

14. The method according to claim 1, applied to retroviruses included in a human or animal genome.

15. The method according to claim 1, applied to a modulation of the immunizing reaction of the organism of a human or animal subject.

16. The method according to claim 1, applied to Ebola virus.

17. The method according to claim 1, wherein immuno-competent cell processing steps are automatized.

18. A system for processing immuno-competent cells in view of an antiviral therapy on a human or animal subject, comprising:
means for collecting immuno-competent cells from said human or animal subject, and
means for processing said collected immuno-competent cells, so as to stimulate at least a part of said collected immuno-competent cells, by presenting to said cells a muted viral particle, in view of a re injection of said stimulated cells into said human or animal subject.

19. The system of claim 18, further comprising:
means for cryogenically preserving collected immuno-competent cells, and
means for defrozing said cryogenically preserved cells, in view of processing said cells.

20. The system of claim 18, further comprising:
means for cryogenically preserving processed and stimulated immuno-competent cells, and
means for defrozing said cryogenically preserved stimulated cells, in view of reinjecting said cells.

21. The system of claim 19, further comprising a cell-bank for storing the preserved immuno-competent cells.

22. An antiviral therapeutic treatment method for a human or animal subject having beforehand been submitted to a collection of immuno-competent cells, comprising a step of injecting into said human or animal subject a selection of immuno-competent cells among said preserved immuno-competent cells, that have been stimulated in vitro by presenting a muted viral antigen.
